(19) United States
(12) Patent Application Publication
(10) Pub. No.: US 2007/0093685 A1
(43) Pub. Date: Apr. 26, 2007

(54) Meditation facilitating device with timer

(76) Inventor: Brian Bighetty, Pukatawagan (CA)

Correspondence Address: Ade & Company Inc., P.O. Box 28006, 1795 Henderson Highway, Winnipeg, MB, R2G IP0, CA

(21) Appl. No.: 11/357,009

(22) Filed: Feb. 21, 2006

(30) Foreign Application Priority Data
Oct. 8, 2005 (CA) 2,515,661

(51) Int. Cl. A61M 21/00

(52) U.S. Cl. 600/26

(57) ABSTRACT

A meditation facilitating device for blocking out light and sound from the surrounding environment during periods of relaxation having a timer for allowing the user to meditate for a predetermined amount of time. The devices features a U or C shaped housing extending about the front of the user's head between the ears, thereby defining an eye portion and two ear portions. The housing is semiflexible so as to engage the users head at the ears during use and be removable by pulling the ear portions apart. A layer of sound absorbing, compressible resilient material is provided between the housing and the users head to increase comfort and further block out light and sound. The timer is set by the user and indicates the end of the session by actuation of an audible alarm positioned to be heard by the user despite the sound absorbing material.

This invention relates to a device worn on a user's head to selectively block out light and sound during mediation, the device having a built-in timer arranged to interrupt a meditation session after a user-controlled period of time.

### BACKGROUND OF THE INVENTION

To obtain optimum relaxation in a meditative state, it is essential to block out the distractions of the surrounding environment. One way to do this is to cover the eyes and ears during meditation in order to minimize audible and visual disturbances. U.S. Pat. No. 6,088,836 discloses a meditation and sleep enhancing device for this purpose. The device features a light occlusion portion that fits over the eyes having a headrest portions extending from one end. The headrest portion is wrapped around the head and attached to the other ends of the light occlusion portion, thereby covering the user's ears. A chin strap extends between the opposite ends of the headrest portion in order to firmly secure the device on the users head. A problem with this invention, and others like it, is that while it successfully blocks out distracting light and sounds for improved meditation, it does not offer a means by which the user can keep track of the amount of time spent in the meditative state without removing the device. To check the time, the user must expose his or her eyes to the external environment, thereby breaking the concentration of the meditative state. The use of an external timer with an audible alarm to track the time is likely futile as the sound will be muffled or blocked out by the headrest portion. As a result, there is a need for a meditation assisting device that allows the user to steadily maintain a relaxed meditative state while not having to worry about missing time sensitive obligations.

### SUMMARY OF THE INVENTION

According to an aspect of the invention there is provided a device comprising:

a housing being adapted to be detachably supported on the head of a user, the housing comprising:
an eye portion for interrupting transmission of light from an environment surrounding the user to eyes of said user, said eye portion being disposed forward of said eyes so as to substantially cover said eyes;
two ear portions for interrupting transmission of sound from the environment surrounding the user to ears of said user, each ear portion being disposed adjacent a respective one of said ears so as to substantially cover said ears; and
a timer element arranged to alert the user that a period of time has elapsed.

The present invention improves upon the prior art by providing a built-in timer to alert the user that a particular amount of time has passed as they meditate. As a result, the user does not have to remove the device to periodically check the time. The user also does not have to worry about missing the alarm of an external timer device due to the light and sound blocking features of the device. The user can achieve a more relaxed state as the meditation is undisturbed and the mind is less preoccupied with concerns over time. Brief meditation sessions can be squeezed into a busy schedule without the risk of missing important deadlines or obligations.

Preferably the period of time is variable and controlled by the user.

Preferably the timer element comprises a dial which is rotatable about an axis thereof by the user, a degree of rotation about said axis corresponding to a respective period of time.

Preferably the timer comprises an audible alarm means arranged to be heard by the user upon elapse of the period of time.

Alternatively the timer may comprise a visual alarm means arranged to be seen by the user upon elapse of the period of time.

Preferably the housing comprises a generally U-shaped headband extending about the head of the user between the ears of said user in such a way as to cover the eyes of the user.

Preferably the headband is tensioned such that the housing detachably engages the ears of the user at so as to be supported on the head thereby.

Preferably the housing further comprises a layer of sound absorbing, compressible resilient material supported on an inside surface of the headband arranged to fit flush against the head of the user so as to prevent light and sound from entering spaces between said headband and said head.

Preferably the timer element is supported on one of the two ear portions.

Each ear portion may comprise movable sound blocking means, said movable sound blocking means arranged for motion between an open position wherein the transmission of sound from the environment to the ears is allowed and a closed position wherein the transmission of sound from the environment to the ears is interrupted by said sound blocking means. In this case, preferably the timer element comprises means to move the movable sound blocking means to the open position upon elapse of the period of time.

Alternatively each ear portion may comprise sound blocking material so as to prevent the sound from the environment from reaching the ears. As a further alternative, each ear portion may comprise sound cancellation means. In this case, the sound cancellation means of each ear portion may comprise:

sound measuring means for measuring sound waves from the environment surrounding the user; and

sound generating means for generating respective sound waves being opposite in phase to the sound waves from the environment such that said respective sound waves cancel out said sound waves from the environment.

In this case, the sound cancellation means can be selectively activated and deactivated by the user to respectively interrupt and allow the transmission of sound from the environment to the ears and the timer element comprises means to deactivate the sound cancellation means upon elapse of the period of time.

The eye portion may comprise movable light blocking means, said movable light blocking means arranged for motion between an open position wherein the transmission of light from the environment to the eyes is allowed and a closed position wherein the transmission of light from the environment to the eyes is interrupted by said light blocking means. In this case, preferably the timer element comprises means to move the movable light blocking means to the open position upon elapse of the period of time.

Alternatively, the eye portion may comprise substantially opaque material. As a further alternative, the eye portion may comprise an eyepiece arranged to be adjustable between substantially opaque & substantially transparent states. In this case, preferably the eye portion further comprises control means by which the user can selectively adjust the eyepiece between the substantially opaque & transparent states and the timer element comprises means to adjust the eyepiece to the substantially transparent state upon elapse of the period of time.

### BRIEF DESCRIPTION OF THE DRAWINGS

In the accompanying drawings, which illustrate exemplary embodiments of the present invention:

FIG. 1 is an isometric view of a meditation facilitating device according a first embodiment of the present invention.

FIG. 2 is a top plan view of the meditation facilitating device according to the first embodiment of the present invention.

FIG. 3 is a front view of the meditation facilitating device according to the first embodiment of the present invention.

FIG. 4 is an isometric view of a meditation facilitating device according to an alternate embodiment of the present invention.

### DETAILED DESCRIPTION

The present invention is a sensory blocking device designed to be worn on the head by a user during periods of intense concentration or relaxation, such as required in meditation practices. The device blocks out visual and audible distractions so that the user can meditate or relax without disturbance. The device includes a timer that can be set in order to meditate for a particular amount of time. The timer has a means to alert the user that the time has expired, at which point they can remove the device or set an additional amount of time and continue meditating. The device adds the element of time management to devices that are intended to block the sight and hearing of the user so that relaxation or meditation can be fit into a busy schedule without concern over the possibility of accidentally allowing the session to run long and missing important obligations.

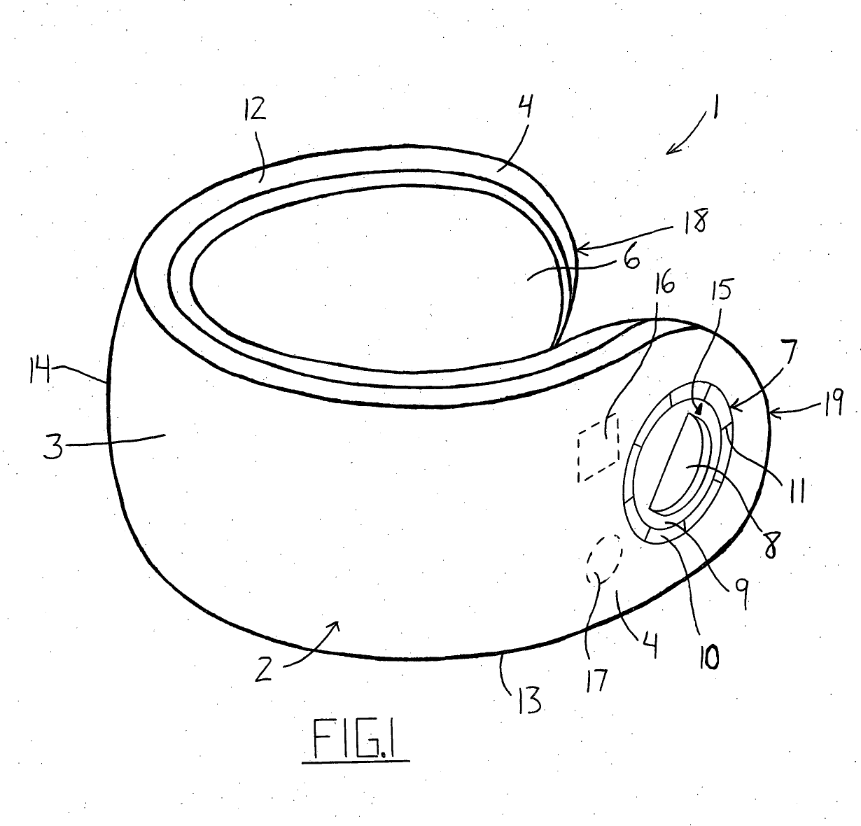

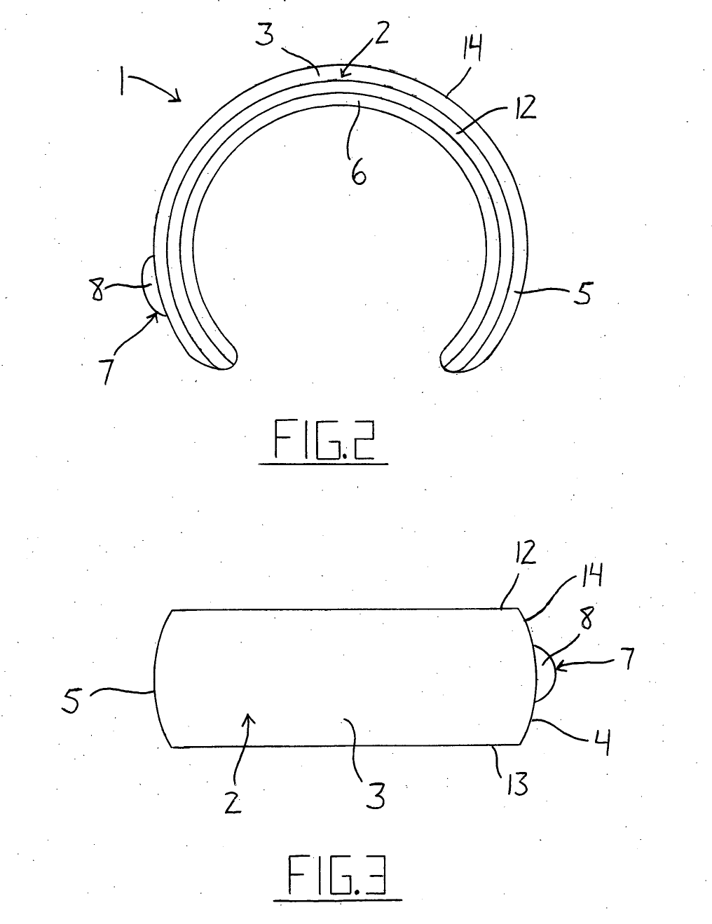

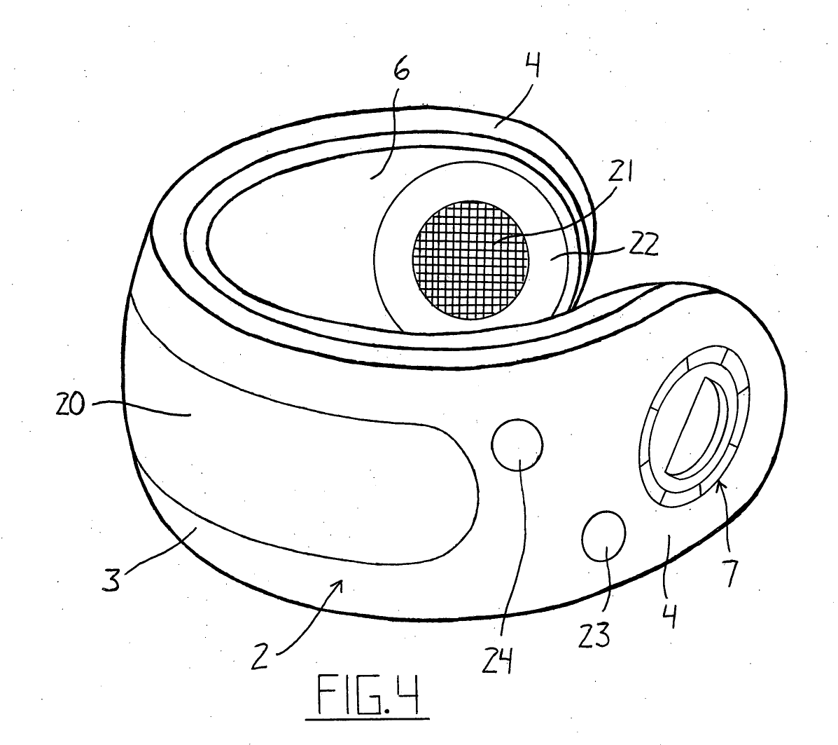

As shown in FIG. 1, the device 1 features housing 2 having an eye portion 3 and two opposing ear portions 4. The device 1 is worn on the head of a user (not shown) such that the ear portions 4 fit over the user's ears with the eye portion 3 extending between the ear portions 4 about the front of the user's head, thereby cover the user's eyes. The housing 3 is made of semiflexible PVC and has a general U or C shape when view from above, as shown in FIG. 2. The ear portions 4 are located at opposite ends 18 and 19 of the U-shaped housing 2 with the eye portion 3 disposed between them. The shape of the housing and the semiflexible nature of the material allows easy removal of the device while ensuring that it is adequately supported when worn on the user's head. The housing's tendancy for the ear portions 4 to move together after being pulled apart ensures firm engagement with the user's ears when worn, so that the device 1 remains on the user's head with the eye portion 3 resting over the user's nose, blocking the eyes. The outer surface 14 of the housing 3 is shaped to curve convexly between flat upper and lower surfaces 12 and 13 to give the device 1 a sleek, appealing appearance.

A layer 6 of compressible, sound absorbing resilient material is provided on the inside surface of the housing 2 in order to ensure the comfort of the user when wearing the device. Held in contact with the user's head by the housing 2, this layer 6 conforms to the shape of the user's head due to the compressibility and resiliency of the material and further provides a soft feel against the user's head. In addition to providing a cushion between the housing 2 and the user's head, this layer 6 serves to improve the sensory blocking function of the device 1. By taking up space between the housing 2 and the features of the user's face, the layer 6 prevents the entry of light from the surrounding environment from above and below. This is necessary as the opaque eye portion 3 of the PVC housing 2 will block light from a wide variety of angles, but cannot block light from well above or below the user as it does not fit flush against the user's eyes. Also, the resilient layer 6 provides further resistance to the transmission of sound from the surrounding environment to the user. The sound absorbing material of the layer 6 prevents sound from reaching the user through the space left between the ear portions 4 of the housing 2 and the user's ears. Additionally, the material will block sound that has already been muffled by the ear portions 4 of the PVC housing. Again, the resiliency and compressibility of the material of the layer 6 ensure an adequate seal, as the material takes the shape of the user's ears.

The device 1 features a built-in timer 7 supported in the housing 2 for alerting the user that a prescribed amount of time has elapsed during meditation or relaxation. The timer 7 includes a disc 9 that is rotatable about its central axis by means of a knob 8 rigidly supported thereon. Indicia 11 are located on a dial 10 positioned around the disc 9 to provide the user with a reference to determine how much to rotate the timer's disc 9 in order to count down a particular amount of time. The user rotates the disc 9 by turning the knob 8 such that a mark 15 on the disc 9 aligns with one of the indicia 11 on the dial 10 corresponding to and visually indicating the length of time for which the user wishes to block out visual and audible distractions from the surrounding environment. The timer includes an audible alarm that is activated when the period of time for which the timer is set expires. While the layer 6 of sound absorbing material is substantially thick so as to absorb the majority of sounds from the outside environment, the audible alarm source 16 is positioned within the layer 6 such that while partially absorbed by the material, a significant portion of the sound emitted by the alarm source 16 will reach the user's ears. At this point, the user can end the session and remove the device 1 or reset the timer 7 for another period of time and continue the session. The timer 7 can be reset by the user without removing the device 1 once the user is familiar with the device, as he or she can associate a degree of rotation of the timer's disc 9 with a particular length of time. With this association in mind, the user can rotate the knob 8 by feel and set the timer 7 with a reasonable amount of accuracy. The timer 7 is of an electronic type in order to avoid the noise created by a spring wound timer. As a result, the timer 7 requires a power supply which is provided in the form of a rechargeable battery 17 mounted with the timer 7 in the housing 2. The small electronic timer 7 retains the true portability of the device while the rechargeable battery 17 reduces environmental hazards.

The above description refers to a single embodiment of the present invention. It should be noted that a number of alternate embodiments are possible, each featuring a housing 2 having an eye portion 3 and two ear portions 4 and a timer 7. The ear portions 4 may feature a means by which the transmission of sound can be selectively interrupted or allowed. This could be provided by an arrangement including openings through the housing 2 and layer 6 at the ear portions 4 through which sound can travel. FIG. 4 shows an alternate embodiment in which openings 21 are provided in a recessed portion 22 of the layer 6 at the ear portions 4. A movable sound blocking means could then be moved between an open position where the openings are unobstructed, allowing sound to pass therethrough, and a closed position in which the openings are blocked by the movable means, preventing sound from reaching the user's ears. Alternatively, sound cancellation technology could be used where the sound waves from the environment are measured and sound waves of opposite phrase are produced in order to cancel out the outside noise and thus prevent it from reaching the user. In either of these cases, a way of providing the user with control over whether sound is allowed to reach the ears or not could be provided. FIG. 4 shows a push button control means 23 for this purpose on the housing 2 between one of the ear portions 4 and the eye portion 3.

Similarly, the eye portion 3 may feature a means by which the transmission of light can be selectively allowed or interrupted. A movable light blocking means, such as an opaque visor or eyepiece, could be provided that moves between an open position where light from the environment is allowed the reach the user's eyes, and a closed position where the light is blocked. Alternatively, an eyepiece could be provided which is adjustable between a substantially opaque state and a substantially transparent state, thereby being capable of either blocking or allowing the transmission of light through it. Such an eyepiece 20 is shown mounted in an opening in the eye portion 3 of the housing 2 in FIG. 4. Again, in either of these cases a means of controlling whether the light is permitted to reach the user's eyes or is blocked before doing so could be provided. FIG. 4 shows a push button control means 24 for this purpose on the housing 2 between one of the ear portions 4 and the eye portion 3.

Different types of timers could also be used. Timer's providing alternative forms of stimulus, such as touch or light, could be used in place of the timer having an audible alarm. A timer using a visual alert would have to be positioned between the user's eyes and the eye portion disposed forward thereof such that the alert can be seen by the user. For example, the alert could be embedded in a recess in the layer of sound absorbing material directly in front of the user's eyes. In a simplified embodiment, the device could include a timer that is not adjustable to a particular length of time by the user, but rather has a timer that counts down a fixed period only. Different styles of timers could be used and be placed in different locations on the housing of the device. Electrical components can be powered with power supplies other than the rechargeable battery discussed above. In addition, a spring wound timer could be used to eliminate the need for a power supply. In this case, the timer should be positioned on the device such that the ticking of the timer cannot be heard by the user as the timer winds down.

Further alternate embodiments could include devices of different materials, shapes or other characteristics with or without a layer of material on the inside surface, having a timer supported on the device such that the user is notified that an amount of time has expired since the device was put on or the timer was last reset. In the aforementioned cases where the sound and/or light blocking means can be selectively activated and deactivated, the timer could be arranged to control one or both of said means such that the disturbances are blocked out until the timer goes off, at which point sound and/or light is allowed to reach the user, signifying that the period of time has expired.

Since various modifications can be made in my invention as herein above described, and many apparently widely different embodiments of same made within the spirit and scope of the claims without department from such spirit and scope, it is intended that all matter contained in the accompanying specification shall be interpreted as illustrative only and not in a limiting sense.

1. A meditation facilitating device comprising:
a housing being adapted to be detachably supported on the head of a user, the housing comprising:
an eye portion for interrupting transmission of light from an environment surrounding the user to eyes of said user, said eye portion being disposed forward of said eyes so as to substantially cover said eyes;
two ear portions for interrupting transmission of sound from the environment surrounding the user to ears of said user, each ear portion being disposed adjacent a respective one of said ears so as to substantially cover said ears; and
a timer element arranged to alert the user that a period of time has elapsed.

2. The device according to claim 1 wherein the period of time is variable and controlled by the user.

3. The device according to claim 1 wherein the timer element comprises a dial which is rotatable about an axis thereof by the user, a degree of rotation about said axis corresponding to a respective period of time.

4. The device according to claim 1 wherein the timer comprises an audible alarm means arranged to be heard by the user upon elapse of the period of time.

5. The device according to claim 1 wherein the timer comprises a visual alarm means arranged to be seen by the user upon elapse of the period of time.

6. The device according to claim 1 wherein each ear portion comprises movable sound blocking means, said movable sound blocking means arranged for motion relative to the housing between an open position wherein the transmission of sound from the environment to the ears is allowed and a closed position wherein the transmission of sound from the environment to the ears is interrupted by said sound blocking means.

7. The device according to claim 1 wherein each ear portion comprises sound blocking material so as to prevent the sound from the environment from reaching the ears.

8. The device according to claim 1 wherein each ear portion comprises sound cancellation means.

9. The device according to claim 8 wherein the sound cancellation means of each ear portion comprises:
sound measuring means for measuring sound waves from the environment surrounding the user; and
sound generating means for generating respective sound waves being opposite in phase to the sound waves from the environment such that said respective sound waves cancel out said sound waves from the environment.

10. The device according to claim 8 wherein the sound cancellation means further comprises control means by which the user can selectively activate and deactivate said sound cancellation means to respectively interrupt and allow the transmission of sound from the environment to the ears.

11. The device according to claim 1 wherein the eye portion comprises movable light blocking means, said movable light blocking means arranged for motion relative to the housing between an open position wherein the transmission of light from the environment to the eyes is allowed and a closed position wherein the transmission of light from the environment to the eyes is interrupted by said light blocking means.

12. The device according to claim 1 wherein the eye portion comprises an eyepiece arranged to be adjustable between substantially opaque & substantially transparent states, said eyepiece being fixed relative to the housing.

13. The device according to claim 12 wherein the eye portion further comprises control means by which the user can selectively adjust the eyepiece between the substantially opaque & transparent states.

14. The device according to claim 1 wherein the housing comprises a generally U-shaped headband extending about the head of the user between the ears of said user in such a way as to cover the eyes of the user, wherein the headband is tensioned such that the housing detachably engages the ears of the user at so as to be supported on the head thereby.

15. The device according to claim 1 wherein the housing further comprises a layer of sound absorbing, compressible resilient material supported on an inside surface of the housing arranged to fit flush against the head of the user so as to prevent light and sound from entering spaces between said housing and said head.

16. The device according to claim 1 wherein the timer element is supported on one of the two ear portions.

17. The device according to claim 6 wherein the timer element comprises means to move the movable sound blocking means to the open position upon elapse of the period of time.

18. The device according to claim 9 wherein the timer element comprises means to deactivate the sound cancellation means upon elapse of the period of time.

19. The device according to claim 11 wherein the timer element comprises means to move the movable light blocking means to the open position upon elapse of the period of time.

20. The device according to claim 13 wherein the timer element comprises means to adjust the eyepiece to the substantially transparent state upon elapse of the period of time.
